(19) United States
(12) Patent Application Publication
(10) Pub. No.: US 2011/0293685 A1
(43) Pub. Date: Dec. 1, 2011

(54) SCAFFOLDS FOR TISSUE ENGINEERING AND REGENERATIVE MEDICINE

(75) Inventors: Catherine K. Kuo, Boston, MA (US); Nathan Zamarripa, Freeport, ME (US); Amelia H. Thomas, Medford, MA (US)

(73) Assignee: TRUSTEES OF TUFTS COLLEGE, Medford, MA (US)

(21) Appl. No.: 13/122,414

(22) PCT Filed: Oct. 5, 2009
(86) PCT No.: PCT/US09/59547

§ 371 Date: Aug. 22, 2011

Related U.S. Application Data
(60) Provisional application No. 61/102,440, filed on Oct. 3, 2008.

(51) Int. Cl. A61K 9/70; A61P 17/00; A61K 35/12

(52) U.S. Cl. 424/422; 424/400; 424/93.7

(57) ABSTRACT

The methods and compositions described herein relate to novel 3-dimensional porous scaffolds useful for tissue re-generation, enhancement, or tissue repair. Electrospinning or other methods are used to create mats comprised of fibers that can be seeded with cells and subsequently rolled into a desired shape/form to replace a desired tissue.

### CROSS-REFERENCE TO RELATED APPLICATIONS

This application claims the benefit of priority under 35 U.S.C. §119(e) of the U.S. Provisional Application No. 61/102,440, filed Oct. 3, 2008, the content of which is incorporated herein by reference in its entirety.

### FIELD OF THE INVENTION

The present invention relates to the field of tissue regeneration and replacement.

### BACKGROUND

Tendons transmit forces from muscle to bone, while ligaments stabilize joint structures. Adult tendons/ligaments (T/L) are similar in structure. Both are comprised of closely packed parallel collagen fiber bundles, composed mainly of collagen type I molecules that are hierarchically organized into structural units. When injured, these tissues are unable to regenerate normally, and current repair strategies are problematic.

Electrospun fiber scaffolds have been demonstrated to be potential substrates for engineered tissues that could replace the damaged tissue. These scaffolds physically resemble the nanofibrous features of the extracellular matrix. The material properties can be tailored for specific applications by controlling variables including chemical composition, fiber diameter and orientation. Fibrous scaffolds have been shown to support stem cell differentiation down the osteogenic, adipogenic, and chondrogenic lineages when cultured in specific differentiation medium. In contrast, differentiation of stem cells down the T/L lineage (tenogenesis) may occur in the absence of specific reagents by responding to aligned orientation coupled with uniaxial tensile stimulation.

### SUMMARY OF THE INVENTION

Described herein are novel 3-dimensional porous scaffolds intended for tissue regeneration or tissue repair. Electrospinning or other methods are used to create mats comprised of fibers with diameters of micrometer or nanometer or other dimension and with fiber orientation that is random, aligned, or any combination thereof. The fibrous material may be comprised of one or more natural materials, or one or more synthetic materials, or a combination of both. When seeded with cells and subsequently rolled into cylindrical form, these constructs can be used to form structures e.g., similar to fascicles of the muscle, tendon, nerve, or ligament.

In one embodiment, these constructs can be used to engineer, enhance, and/or regenerate fascicles of muscle, tendon, or ligament. When grouped together, these fascicular-like constructs will represent tissue constructs that when cultured in vitro or implanted in vivo will support cell adhesion, growth and regenerate tissues such as, for instance and without limitation muscle, tendon, ligament or other connective tissue. Compositions described herein mimic native tissue structure by forming individual engineered fascicles, which are considered sub-components of whole muscle, nerve, tendon, and ligament tissues, and by forming whole tissues comprised of bundled engineered fascicles.

One aspect described herein is a composition comprising: a porous scaffold sheet of fibrous material; and living cells deposited thereupon; wherein the sheet is spirally wound in a jelly-roll like manner. In one embodiment, the cells are eukaryotic cells.

In one embodiment of this aspect and all other aspects described herein, the composition comprises a plurality of the spirally wound structures.

In another embodiment of this aspect and all other aspects described herein, the plurality of spirally wound structures are aligned substantially parallel to, and in contact with each other or separated by sheaths, along a common axis, to form a bundle of the structures.

In another embodiment of this aspect and all other aspects described herein, the fibrous material comprises individual fibers.

In another embodiment of this aspect and all other aspects described herein, the fibers are microfibers or nanofibers.

In another embodiment of this aspect and all other aspects described herein, the fibrous material comprises fibers that are aligned in one direction, are randomly aligned, or any combination thereof. In another embodiment of this aspect and all other aspects described herein, the fibrous material is braided, twisted, or otherwise manipulated to be grouped together or to stand individually.

In another embodiment of this aspect and all other aspects described herein, wherein the fibrous material comprises a natural fiber, a synthetic fiber, a flexible metal fiber, or a combination thereof. In another embodiment, the fibrous material comprises a metal or ceramic particle incorporated into or onto the polymer fibers.

In another embodiment of this aspect and all other aspects described herein, the natural fiber is selected from the group consisting of collagen, fibrin, silk, thrombin, chitosan, chitin, alginic acid, hyaluronic acid, and gelatin.

In another embodiment of this aspect and all other aspects described herein, the synthetic fiber is selected from the group consisting of: representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate).

In another embodiment of this aspect and all other aspects described herein, the cell is selected from the group consisting of stem cells, osteoblasts, myoblasts, neuroblasts, fibroblasts, glioblasts, germ cells, hepatocytes, chondrocytes, keratinocytes, smooth muscle cells, cardiac muscle cells, connective tissue cells, epithelial cells, endothelial cells, hormone-secreting cells, neurons, tenocytes, skeletal myocytes, and skeletal myoblasts.

In another embodiment of this aspect and all other aspects described herein, the stem cell comprises an adult stem cell, an embryonic stem cell or a reprogrammed stem cell.

In another embodiment of this aspect and all other aspects described herein, the composition further comprises a bioactive agent.

In another embodiment of this aspect and all other aspects described herein, the bioactive agent comprises small molecules, proteins, polypeptides, or nucleic acids. The bioactive agent can be a synthetic agent, a natural agent, a compound, or a drug.

Another aspect described herein is a method for producing a tissue construct, the method comprising: contacting a scaffold sheet of fibrous material with a cell; and rolling the scaffold sheet in a jelly-roll like manner to form a spirally wound tissue construct.

In one embodiment of this aspect and all other aspects described herein, the method further comprises aligning a plurality of the spirally wound tissue constructs substantially parallel to, and in contact with each other (or optionally separated by a sheath), along a common axis, to form a bundle of the constructs. Alternatively, the individual jelly-rolls or bundles of rolls can be twisted, braided etc. and held together with sheaths. A sheath can hold sub-bundles of one or more jelly rolls.

Another aspect described herein is a method for replacing or enhancing a tissue, the method comprising: (a) forming a tissue construct by contacting a scaffold sheet of fibrous material with a cell; and rolling the scaffold sheet in a jelly-roll like manner to form a spirally wound tissue construct, (b) implanting the spirally wound tissue construct into a subject in need of tissue replacement or regeneration, wherein the spirally wound tissue construct replaces a tissue.

In one embodiment of this aspect and all other aspects described herein, the tissue is selected from the group consisting of a muscle, a nerve, a ligament, a tendon, or another tissue.

### Definitions

As used herein, “scaffold” refers to a structure, comprising a biocompatible material, that provides a surface suitable for adherence of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### BRIEF DESCRIPTION OF THE FIGURES

FIG. 1. is a schematic showing fabrication of a tissue construct.

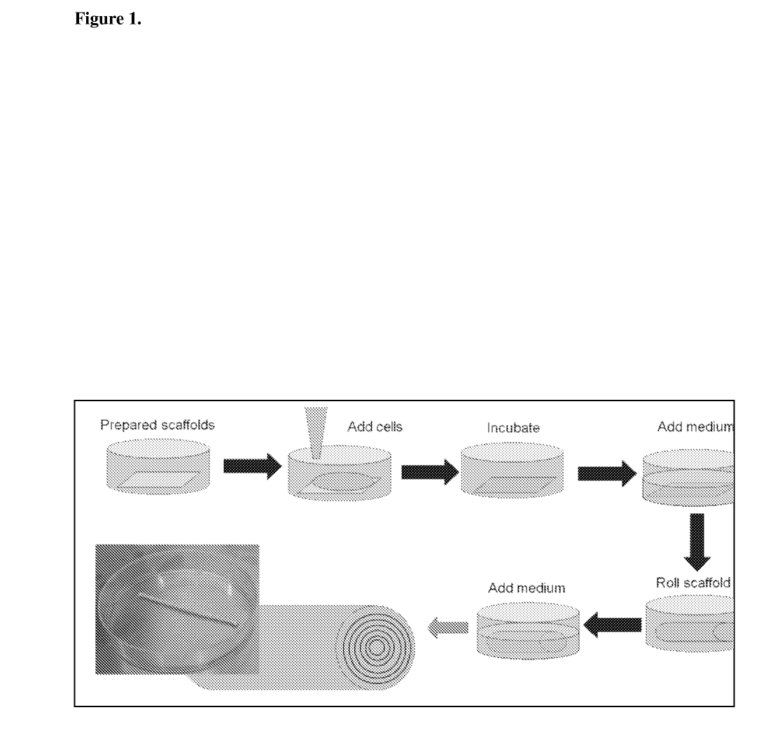

FIG. 2. is a schematic showing three tissue constructs bundled together to form a whole tissue construct.

FIG. 3. is a diagram depicting an exemplary method for making random or aligned fibers in a scaffold mat.

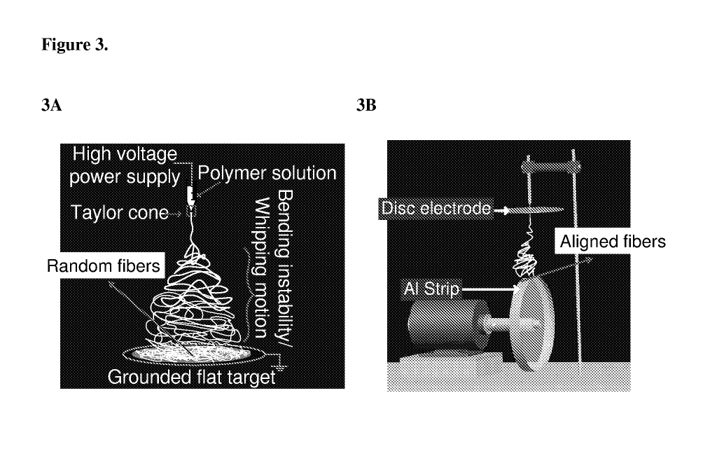

FIG. 4. is a set of micrographs depicting FE-SEM images of aligned nanofiber scaffolds.

FIG. 5. FIG. 5a shows a FE-SEM image of electrospun nanofiber scaffold with aligned morphology seeded with mesenchymal progenitor cells, rolled, and cultured for 24 h; FIG. 5b shows a rolled cell-scaffold construct that has been fixed and stained with DAPI to visualize cell distribution.

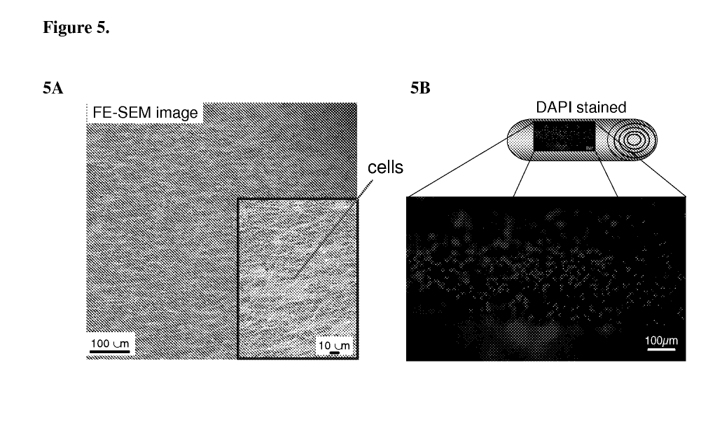

FIG. 6. is a diagram depicting an exemplary method to hold scaffolds in place.

### DETAILED DESCRIPTION

Described herein are novel 3-dimensional porous scaffold compositions, that can be implanted into a subject for tissue regeneration, tissue enhancement, or tissue repair. Scaffold sheets comprised of fibers with diameters of micrometer or nanometer or other dimension and with fibers that are oriented at random, aligned, etc. are produced by e.g., electrospinning. The fibrous material may be comprised of natural materials, synthetic materials, flexible metal fibers, or any combination thereof. The compositions are seeded with cells and subsequently rolled into cylindrical form, to form structures e.g., that mimic that of fascicles of the muscle, tendon, nerve, or ligament. In addition, a plurality of compositions can be bundled together to form fascicular-like constructs that when cultured in vitro or implanted in vivo will support cell adhesion, growth, differentiation and/or regenerate tissues such as, for instance and without limitation muscle, tendon, ligament, nerve or other tissue.

### Fibrous Materials

Essentially any fibrous scaffolding material can be used with the methods and compositions described herein, as long as the material is biocompatible with cells (i.e., does not induce cell death, permits cell growth and differentiation). When the composition is intended to be implanted into a subject, it is preferred that the material does not cause an inflammatory reaction or immune response in the subject.

Biocompatible polymers useful in the present invention include, for example, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), silk fibroin (WO 2005/012606), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419). Representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate) can also be used in the methods and compositions described herein. The above-noted polymers are non-limiting examples, and other biocompatible polymers known to those of skill in the art are also contemplated for use with the methods and compositions described herein.

In addition, polymer materials can be modified or combined with other material types (e.g., ceramic particles) which can be incorporated during or after scaffold formation.

### Scaffolds

Scaffolds for use in the instant invention are made from biocompatible materials. Some non-limiting examples of biocompatible materials include silk, collagen, or other protein-based polymers. These materials can be modified or combined with other material types (e.g., ceramic) during or after scaffold formation.

The ideal properties of the biocompatible materials for use in the instant invention include: mechanical integrity, thermal stability, ability to self-assemble, non-immunogenic, bioresorbable, slow degradation rate, capacity to be functionalized with, for instance, cell growth factors, and plasticity in terms of processing into different structural formats.

Scaffolds for use in the instant invention may be any structural format including, for example, nanoscale diameter fibers from electrospinning, fiber bundles and films. Methods of forming these various formats from fibrous materials (e.g., silk) are known to the skilled artisan. See, for instance, Jin et al., 2002, Biomacromolecules 3:1233-1239; Jin et al., 2004, Biomacromolecules 5:711-717; Altman et al., 2002, Biomaterials 23:4131-4141; Altman et al., 2002, J Biomech. Eng. 124:742-749; and Meinel et al., 2004, Biotechnol. Bioeng. 88:379-391, and U.S. Patent Publication No. 20050260706, each incorporated herein by reference in its entirety.

There are numerous ways known to the skilled artisan for making porous scaffolds, including freeze-drying, salt leaching and gas foaming (Nazarov et al, 2004, Biomacromolecules 5:718-726, incorporated herein by reference in its entirety). When the desired scaffold properties are high porosity and very high compressive strength, gas foaming may be preferred. When the desired scaffold properties are high porosity and low compressive strength, the freeze-dried scaffolds may be preferred. For the scaffolds used in alleviating or treating a bone defect, the preferred method of making the scaffold is salt leaching. The salt leaching method is preferably an all-aqueous method when avoidance of organic solvents is necessary. Salt leaching methods yield scaffolds having high porosity and high compressive strength. The pores are preferably homogenous and interconnected. Pore size in the scaffold is determined by the size of the salt particles used in the salt leaching process. Larger salt particles yield larger pores in the silk scaffold. Preferably the pores are about 50 to about 1200 microns, more preferably about 250 to about 1100 microns and more preferably about 450 to about 1000 microns. Preferred compressive strength for the scaffold is at least about 250 KPa, more preferably at least about 300 KPa and more preferably about 320 KPa. Preferred modulus is about 2800 to about 4000 KPa, more preferably about 3000 to about 3750 KPa and most preferably about 3200 to about 3500 KPa. Scaffolds may be sterilized by autoclaving them, treatment with ethylene oxide gas or with alcohol.

The scaffolds of the instant invention may be modified with one or more molecules. Any molecule may be attached, covalently or non-covalently, to the biomaterial to modify it. For instance, cell growth factors may be covalently bound to the scaffold material. Alternatively, a tissue construct may be coated with a molecule. Molecules for modification are preferably non-immunogenic in the intended recipient individual. A molecule whose sequence is native to the intended recipient individual is considered to be non-immunogenic. Preferred molecules for modification are molecules that function in controlling cell attachment, cell differentiation and cell signaling. Non-limiting examples of such molecules include the integrin binding tripeptide RGD, parathyroid hormone (PTH) and BMP-2.

### Production of Fibers

Fibers may be produced using any method known in the art such as, melt spinning, extrusion, drawing, wet spinning or electrospinning. Alternatively, as the concentrated solution has a gel-like consistency, a fiber can be pulled directly from the solution.

In one embodiment, the fibers are produced using electrospinning. Electrospinning can be performed by any means known in the art (see, for example, U.S. Pat. No. 6,110,590). Preferably, a steel capillary tube with a 1.0 mm internal diameter tip is mounted on an adjustable, electrically insulated stand. Preferably, the capillary tube is maintained at a high electric potential and mounted in the parallel plate geometry. The capillary tube is preferably connected to a syringe filled with fibrous scaffold material solution. Preferably, a constant volume flow rate is maintained using a syringe pump, set to keep the solution at the tip of the tube without dripping. The electric potential, solution flow rate, and the distance between the capillary tip and the collection screen are adjusted so that a stable jet is obtained. Dry or wet fibers are collected by varying the distance between the capillary tip and the collection screen.

A collection screen suitable for collecting fibrous scaffold material fibers can be a wire mesh, a polymeric mesh, or a water bath. Alternatively and preferably, the collection screen is an aluminum foil. The aluminum foil can be coated with Teflon fluid to make peeling off the fibrous scaffold material fibers easier. One skilled in the art will be able to readily select other means of collecting the fiber solution as it travels through the electric field. The electric potential difference between the capillary tip and the aluminum foil counter electrode is, preferably, gradually increased to about 12 kV, however, one skilled in the art can adjust the electric potential to achieve suitable jet stream.

Electrospinning for the formation of fine fibers has been actively explored recently for applications such as high performance filters and biomaterial scaffolds for cell growth, vascular grafts, wound dressings or tissue engineering. Fibers with a nanoscale diameter provide benefits due to their high surface area. In this electrostatic technique, a strong electric field is generated between a polymer solution contained in a syringe with a capillary tip and a metallic collection screen. When the voltage reaches a critical value, the charge overcomes the surface tension of the deformed drop of suspended polymer solution formed on the tip of the syringe, and a jet is produced. The electrically charged jet undergoes a series of electrically induced bending instabilities during passage to the collection screen that results in stretching. This stretching process is accompanied by the rapid evaporation of the solvent and results in a reduction in the diameter of the jet. The dry fibers accumulated on the surface of the collection screen form a non-woven mesh of nanometer to micrometer diameter fibers even when operating with aqueous solutions at ambient temperature and pressure. The electrospinning process can be adjusted to control fiber diameter by varying the charge density and polymer solution concentration, while the duration of electrospinning controls the thickness of the deposited mesh.

Protein fiber spinning in nature, such as for silkworm and spider silks, is based on the formation of concentrated solutions of metastable lyotropic phases that are then forced through small spinnerets into air. The fiber diameters produced in these natural spinning processes range from tens of microns in the case of silkworm silk to microns to submicron in the case of spider silks. The production of fibers from protein solutions has typically relied upon the use of wet or dry spinning processes. Electrospinning offers an alternative approach to protein fiber formation that can potentially generate very fine fibers. This can be a useful feature based on the potential role of these types of fibers in some applications such as biomaterials and tissue engineering.

Fibers or fiber bundles can be braided, twisted, or manipulated by one of skill in the art to be grouped together or stand individually for the formation of scaffolds. One of skill in the art can form scaffolds using any configuration of fibers that is desired (e.g., aligned fibers, braided, twisted, random etc.).

### Fasteners for Holding Scaffolds Together

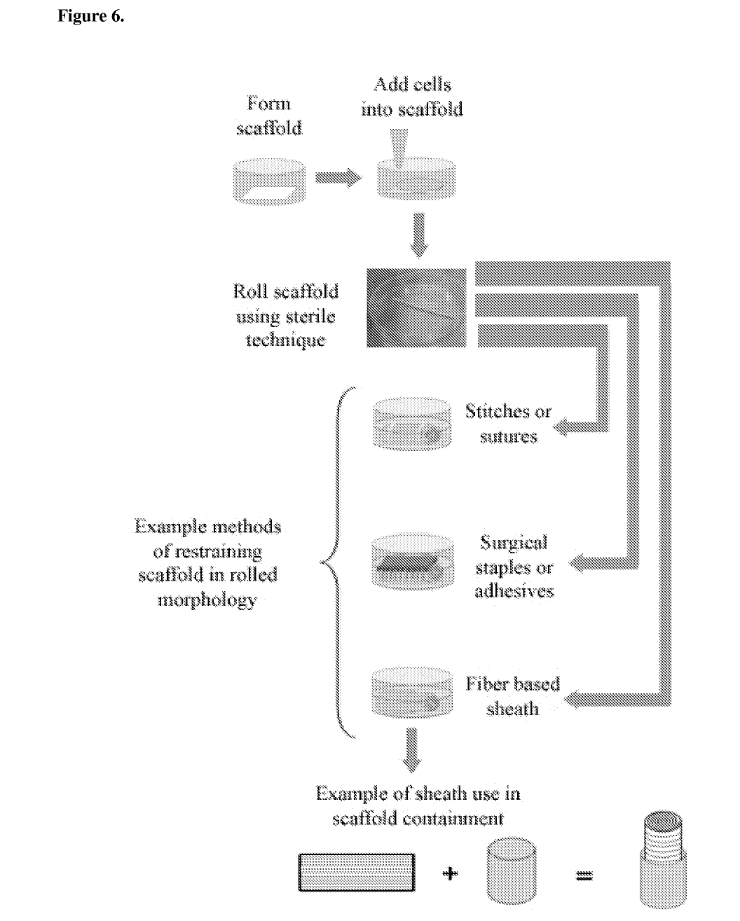

A scaffold can be held together into any shape and/or size to be used as a construct for tissue regeneration, enhancement, or repair of a desired tissue. For example, in one embodiment the scaffold is rolled in a jelly-roll like manner to produce a cylindrical form. Once rolled, the scaffolds can be held in place using various techniques to improve the scaffold's structural integrity and durability. For example, a rolled scaffold can be effectively secured from unfolding by suturing the scaffold ends, employing a fiber based scaffold sheath modeled after natural tissue sheaths (FIG. 6), or by the use of a fastener.

A “fastener” is used to maintain the tissue construct in a desired shape for implantation. Some non-limiting examples of fasteners include staples, sutures, pins, sheaths (e.g., fibrous sheath), tissue glue, biocompatible epoxies etc, or any combination thereof. In one embodiment, the sheath is a nanofiber based sheath.

A fastener can be used when the construct is unable to maintain a desired shape (e.g., cylindrical) or a desired size. Alternatively, a fastener can be used to mimic natural tissues, which often have a sheath in the body. Fasteners can be biodegradable such that they dissolve or degrade over time following implantation or alternatively permanent fasteners can be used. One of skill in the art can determine the appropriate fastener to be used based on the tissue type and the amount of support necessary to maintain a tissue construct in a desired form.

The use of fasteners permits a scaffold to be formed in any shape and thus can be used to promote regeneration or repair of essentially any tissue. For example, scaffolds can be formed that replace/repair muscle, ligament, tendon, connective tissue, nerves, nerve channels, complex organs (e.g., liver, pancreas etc), endothelial tissue, gastrointestinal tissue, cardiac tissue and/or ducts (e.g., bile ducts). One of skill in the art can design and/or prepare scaffold constructs for any desired tissue type, tissue size, and/or tissue shape. It is further contemplated that a construct can be “custom fit” to correspond with the size of a particular subject (e.g., child, youth, adult etc.).

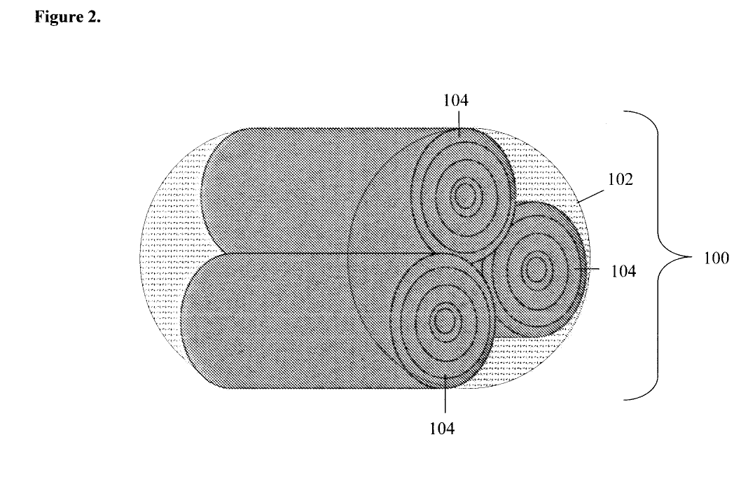

Fasteners can also be used to connect a plurality of constructs together (e.g., at least 2, at least 3, at least 4, at least 5, at least 6, at least 7, at least 8, at least 9, at least 10, at least 20, or more). The constructs can be fastened together in any desired shape and can e.g., be aligned along a common axis, perpendicular to one another, or in any other configuration as desired by one of skill in the art. In one embodiment, as shown herein in FIG. 2, at least two cylindrical constructs (104) are fastened together along a common axis to form a whole tissue construct (100). In one embodiment, as illustrated in FIG. 2, the constructs are enclosed by a fibrous sheath (102). Furthermore, a fastener can be chosen to mimic an endogenous state of a tissue. For example, a fibrous sheath (102) can be used as fasteners for tendon-shaped scaffolds, which is similar to the sheath found surrounding tendons in vivo.

In one embodiment, the fastener is a sheath. A sheath can (1) hold individual rolled scaffolds together, (2) hold groups of rolled scaffolds together, and/or (3) mimic natural sheath structure found around individual bundles and groups of bundles.

Individual jelly-rolls or bundles of rolls can be twisted, braided, etc. and can be held together with sheaths. Thus, sheaths can hold sub-bundles of one or more jelly-rolls.

### Bioactive Agents

Additives suitable for use with the present invention include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January;60(1):119-32; Hersel U. et al. 2003 Biomaterials. November;24(24):4385-415); biologically active ligands; and substances that enhance or exclude particular varieties of cellular or tissue ingrowth. Other examples of additive agents that enhance proliferation or differentiation include, but are not limited to, bone morphogenic proteins (BMP); cytokines, growth factors such as epidermal growth factor (EGF), platelet-derived growth factor (PDGF), insulin-like growth factor (IGF-I and II), TGF-β, and the like. As used herein, the term additive also encompasses antibodies, DNA, RNA, modified RNA/protein composites, glycogens or other sugars, and alcohols.

In another embodiment of the present invention, tissue constructs can contain therapeutic agents. To form these materials, the fibrous material solution can be mixed with a therapeutic agent prior to forming the material or loaded into the material after it is formed. The variety of different therapeutic agents that can be used in conjunction with the biomaterials of the present invention is vast and includes small molecules, proteins, synthetic agents, natural agents, drugs, compounds, peptides and nucleic acids. In general, therapeutic agents which may be administered via the invention include, without limitation: antiinfectives such as antibiotics and antiviral agents; chemotherapeutic agents (i.e. anticancer agents); anti-rejection agents; analgesics and analgesic combinations; anti-inflammatory agents; hormones such as steroids; growth factors (bone morphogenic proteins (i.e. BMP's 1-7), bone morphogenic-like proteins (i.e. GFD-5, GFD-7 and GFD-8), epidermal growth factor (EGF), fibroblast growth factor (i.e. FGF 1-9), platelet derived growth factor (PDGF), insulin like growth factor (IGF-I and IGF-II), transforming growth factors (i.e. TGF-.beta.-III), vascular endothelial growth factor (VEGF)); anti-angiogenic proteins such as endostatin, and other naturally derived or genetically engineered proteins, polysaccharides, glycoproteins, or lipoproteins. Additionally, the fibrous materials described herein can be used to deliver any type of molecular compound, such as, pharmacological materials, vitamins, sedatives, steroids, hypnotics, antibiotics, chemotherapeutic agents, prostaglandins, and radiopharmaceuticals. The methods and compositions described herein are suitable for delivery of the above materials and others including but not limited to proteins, peptides, nucleotides, carbohydrates, simple sugars, cells, genes, anti-thrombotics, anti-metabolics, growth factor inhibitor, growth promoters, anticoagulants, antimitotics, fibrinolytics, anti-inflammatory steroids, and monoclonal antibodies.

### Cells

A number of different cell types or combinations thereof may be employed in the methods and compositions described herein, depending upon the intended function of the tissue engineered construct being produced. In one embodiment, the cells are eukaryotic cells. Exemplary cell types include, but are not limited to: smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells. For example, smooth muscle cells and endothelial cells may be employed for muscular, tubular constructs, e.g., constructs intended as vascular, esophageal, intestinal, rectal, or ureteral constructs; chondrocytes may be employed in cartilaginous constructs; cardiac muscle cells may be employed in heart constructs; hepatocytes and bile duct cells may be employed in liver constructs; epithelial, endothelial, fibroblast, and nerve cells may be employed in constructs intended to function as replacements or enhancements for any of the wide variety of tissue types that contain these cells. In general, any cells may be employed that are found in the natural tissue to which the construct is intended to correspond. In addition, progenitor cells, such as myoblasts or stem cells, may be employed to produce their corresponding differentiated cell types. Stem cells can be adult stem cells, embryonic stem cells, or reprogrammed stem cells. In some instances it may be preferred to use neonatal cells or tumor cells. In one embodiment, the cells are mammalian cells.

Cells can be obtained from donors (allogenic) or from recipients (autologous). Cells can also be of established cell culture lines, or even cells that have undergone genetic engineering. Pieces of tissue can also be used, which may provide a number of different cell types in the same structure.

Appropriate growth conditions for cells (e.g., mammalian cells) are well known in the art (Freshney, R. I. (2000) Culture of Animal Cells, a Manual of Basic Technique. Hoboken N.J., John Wiley & Sons; Lanza et al. Principles of Tissue Engineering, Academic Press; 2nd edition May 15, 2000; and Lanza & Atala, Methods of Tissue Engineering Academic Press; 1st edition October 2001). Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS) or calf serum and 100 U/mI penicillin are appropriate as are various other standard media well known to those in the art. Growth conditions will vary dependent on the type of cells in use and tissue desired.

In one preferred embodiment, tissues and organs are generated for humans. In other embodiments, tissues and organs are generated for animals such as, dogs, cats, horses, lizards, monkeys, or any other animal. In one embodiment, the animal is a mammal, however the methods and compositions described herein are useful with respect to any animal.

The cells that are used for methods of the present invention should be derived from a source that is compatible with the intended recipient. The cells are dissociated using standard techniques and seeded onto and into the scaffold. In vitro culturing optionally may be performed prior to implantation. Methods and reagents for culturing cells in vitro and implantation of a tissue scaffold are known to those skilled in the art.

Cells can be incorporated into a scaffold during the scaffold fabrication process or alternatively, cells are seeded onto/into the scaffolds following scaffold preparation.

Uniform seeding of cells on the fibrous material is preferable. In theory, the number of cells seeded does not limit the final tissue produced, however optimal seeding may increase the rate of generation. The number of seeded cells can be optimized using dynamic seeding (Vunjak-Novakovic et al. Biotechnology Progress 1998; Radisic et al. Biotechnoloy and Bioengineering 2003).

With the methods and compositions described herein organized tissue with a predetermined form and structure can be produced in vitro or in vivo. For example, tissue that is produced ex vivo is functional from the start and can be used as an in vivo implant.

All biomaterials of the present invention may be sterilized using conventional sterilization process such as radiation based sterilization (i.e. gamma-ray), chemical based sterilization (ethylene oxide), autoclaving, or other appropriate procedures. After sterilization the biomaterials may be packaged in an appropriate sterilize moisture resistant package for shipment and use in hospitals and other health care facilities.

### Replacing/Enhancing a Tissue

The compositions described herein can be used for organ repair, replacement or regeneration strategies that may benefit from these unique scaffolds, including but are not limited to, spine disc, cranial tissue, dura, nerve tissue, liver, pancreas, kidney, bladder, spleen, cardiac muscle, skeletal muscle, tendons, ligaments and breast tissues. In one embodiment, the compositions are used for muscle, tendon, ligament or nerve repair, replacement or regeneration strategies.

The scaffolds are shaped into articles for tissue engineering and tissue guided regeneration applications, including reconstructive surgery. The scaffolds may be molded to form external scaffolding for the support of in vitro culturing of cells for the creation of external support organs. In the reconstruction of structural tissues like cartilage and bone, tissue shape is integral to function, requiring the molding of the scaffold into articles of varying thickness and shape. Any crevices, apertures or refinements desired in the three-dimensional structure can be created by removing portions of the matrix with scissors, a scalpel, a laser beam or any other cutting instrument.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All references cited herein are incorporated by reference in their entirety.

The present invention may be as defined in any one of the following numbered paragraphs.

1. A composition comprising: a porous scaffold sheet of fibrous material; and living cells deposited thereupon; wherein the sheet is spirally wound in a jelly-roll like manner.

2. The composition of paragraph 1, comprising a plurality of the spirally wound structures.

3. The composition of paragraph 2, wherein the plurality of spirally wound structures are aligned substantially parallel to each other along a common axis, to form a bundle of the structures.

4. The composition of paragraph 3, wherein the plurality of spirally wound structures are braided, twisted or held together by a sheath.

5. The composition of paragraph 1, wherein the fibrous material comprises individual fibers.

6. The composition of paragraph 5, wherein the fibers are microfibers or nanofibers.

7. The composition of paragraph 1, wherein the fibrous material comprises fibers that are aligned in one direction, randomly aligned, braided, twisted, or any combination thereof.

8. The composition of paragraph 1, wherein the fibrous material comprises a natural fiber, a synthetic fiber, or a combination thereof.

9. The composition of paragraph 8, wherein the natural fiber is selected from the group consisting of collagen, fibrin, silk, thrombin, chitosan, chitin, alginic acid, hyaluronic acid, and gelatin.

10. The composition of paragraph 8, wherein the synthetic fiber comprises two or more polymers.

11. The composition of paragraph 8, wherein the synthetic fiber is selected from the group consisting of: representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate).

12. The composition of paragraph 1, wherein the cell is selected from the group consisting of stem cells, osteoblasts, myoblasts, neuroblasts, fibroblasts, glioblasts, germ cells, hepatocytes, chondrocytes, keratinocytes, smooth muscle cells, cardiac muscle cells, connective tissue cells, epithelial cells, endothelial cells, hormone-secreting cells, neurons, tenocytes, skeletal myocytes, and skeletal myoblasts.

13. The composition of paragraph 12, wherein the stem cell comprises an adult stem cell, an embryonic stem cell or a reprogrammed stem cell.

14. The composition of paragraph 1, wherein the cell is deposited onto or into the scaffold prior to or after spirally winding of the scaffold sheet.

15. The composition of paragraph 1, further comprising a bioactive agent.

16. The composition of paragraph 15, wherein the bioactive agent comprises small molecules, proteins, compounds, drugs, synthetic agents, natural agents, polypeptides, or nucleic acids.

17. A method for producing a tissue construct, the method comprising: contacting a scaffold sheet of fibrous material with a cell; and rolling the scaffold sheet in a jelly-roll like manner to form a spirally wound tissue construct.

18. The method of paragraph 17, further comprising aligning a plurality of the spirally wound tissue constructs substantially parallel to each other along a common axis, to form a bundle of the constructs.

19. The method of paragraph 18, wherein the plurality of spirally wound structures are braided, twisted or held together by a sheath.

20. The method of paragraph 17, wherein the fibrous material comprises a natural fiber, a synthetic fiber, or a combination thereof.

21. The method of paragraph 20, wherein the natural fiber is selected from the group consisting of collagen, fibrin, silk, thrombin, chitosan, chitin, alginic acid, hyaluronic acid, and gelatin.

22. The method of paragraph 20, wherein the synthetic fiber comprises two or more polymers.

23. The method of paragraph 20, wherein the synthetic fiber is selected from the group consisting of: representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate).

24. The method of paragraph 17, wherein the cell is selected from the group consisting of stem cells, osteoblasts, myoblasts, neuroblasts, fibroblasts, glioblasts, germ cells, hepatocytes, chondrocytes, keratinocytes, smooth muscle cells, cardiac muscle cells, connective tissue cells, epithelial cells, endothelial cells, hormone-secreting cells, neurons, tenocytes, skeletal myocytes, and skeletal myoblasts.

25. The method of paragraph 24, wherein the stem cell comprises an adult stem cell, an embryonic stem cell or a reprogrammed stem cell.

26. The method of paragraph 17, wherein the contacting a scaffold sheet with a cell is performed prior to or after the rolling.

27. A method for replacing or enhancing a tissue, the method comprising:
(a) forming a tissue construct by contacting a scaffold sheet of fibrous material with a cell; and rolling the scaffold sheet in a jelly-roll like manner to form a spirally wound tissue construct,
(b) implanting the spirally wound tissue construct into a subject in need of tissue replacement or regeneration,
   wherein the spirally wound tissue construct replaces a tissue.

28. The method of paragraph 27, further comprising aligning a plurality of the spirally wound tissue constructs substantially parallel to each other along a common axis, to form a bundle of the constructs prior to the implanting step.

29. The method of paragraph 27, wherein the plurality of spirally wound structures are braided, twisted or held together by a sheath. 30. The method of paragraph 27, wherein the fibrous material comprises a natural fiber, a synthetic fiber, or a combination thereof.

31. The method of paragraph 30, wherein the natural fiber is selected from the group consisting of collagen, fibrin, silk, thrombin, chitosan, chitin, alginic acid, hyaluronic acid, and gelatin.

32. The method of paragraph 30, wherein the synthetic fiber comprises two or more polymers.

33. The method of paragraph 30, wherein the synthetic fiber is selected from the group consisting of: representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate).

34. The method of paragraph 27, wherein the cell is selected from the group consisting of stem cells, osteoblasts, myoblasts, neuroblasts, fibroblasts, glioblasts, germ cells, hepatocytes, chondrocytes, keratinocytes, smooth muscle cells, cardiac muscle cells, connective tissue cells, epithelial cells, endothelial cells, hormone-secreting cells, neurons, tenocytes, skeletal myocytes, and skeletal myoblasts.

35. The method of paragraph 34, wherein the stem cell comprises an adult stem cell, an embryonic stem cell or a reprogrammed stem cell.

36. The method of paragraph 27, wherein the tissue is selected from the group consisting of a muscle, a ligament, a tendon, a nerve, or other tissue.

37. The method of paragraph 27, wherein the tissue is nerve tissue.

38. The method of paragraph 27, wherein the contacting a scaffold sheet with a cell is performed prior to or after the rolling.

### EXAMPLES

Described herein are methods and compositions using aligned nanofiber materials as scaffolds for engineering tendon or ligament tissues with stem/progenitor cells. Described herein is a novel strategy to produce a cell-integrative biomimetic scaffold for tenogenesis and tendon/ligament engineering. Aligned nanofiber scaffolds were seeded with progenitor cells and subsequently rolled with the leading edge parallel to the axis of alignment. This aligned nanofiber scaffold locally mimicked the native microstructure of tendon/ligament (aligned collagen fibrils), globally mimicked a fascicle or bundle in tendon/ligament tissue, and resulted in cell seeding throughout the construct. This design avoids poor cell seeding throughout the scaffold that is a major limitation of aligned scaffolds due to their low porosity as a result of closely packed aligned fibers.

### Scaffold Fabrication

Electrospun polycaprolactone (PCL) (10% w/v) in chloroform:methanol 2:1 (v:v); PCL:collagen type I (Col I) blend (8% w/v) at 11:5 (w:w) in 1,1,1,3,3,3 hexafluoro-2-propanol (HFIP); and Col I (8% wt) in HFIP were prepared.

Two different target geometries used to control nanofiber orientation: flat target for random orientation; and rotating target for aligned orientation

### Scaffold Preparation, Cell Seeding, and Construct Formation

Primary mesenchymal progenitor cells were isolated, expanded and seeded at 40,000 cells per cm² onto an electrospun scaffold. Scaffolds were cultured for 18 hours at 37° C. in a highly concentrated cell suspension to facilitate cell seeding. After cell adhesion was achieved, medium was added and the cell-seeded scaffolds were cultured for an additional 24 hours at 37° C. Post-incubation, the constructs were rolled along the major axis of alignment and cultured for another 24 hours

### Assays

Scaffold architecture was examined using FE-SEM. Cell adhesion and distribution were observed using DAPI staining.

### Results

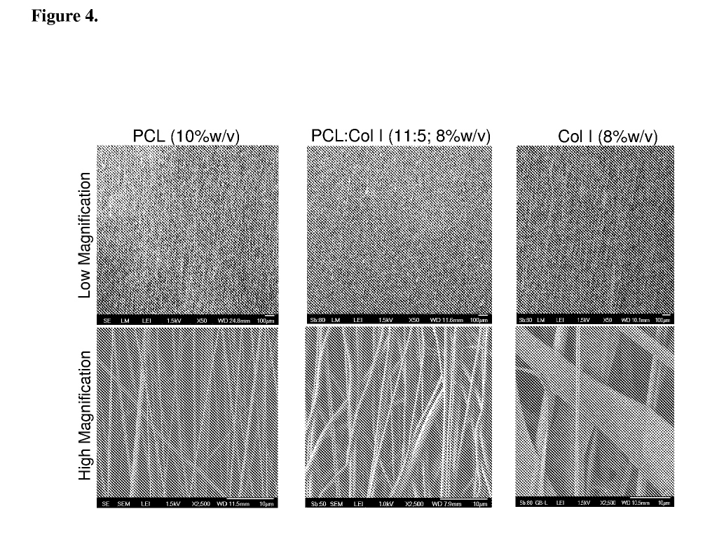

FE-SEM images of PCL, PCL:Col I, and Col I aligned nanofiber scaffolds were compared in FIG. 4. Col I is desirable as a scaffold material because it is a major component of native tendon/ligament and facilitates cell adhesion, but electrospun Col I nanofibers displayed uncontrolled fiber morphology (flat and tape-like) and heterogeneous distribution in fiber diameter. FE-SEM images and visualized DAPI-stained constructs demonstrated uniform distribution of mesenchymal progenitor cells on the surfaces of the nanofiber scaffolds

Rolled scaffolds supported cell adhesion and distribution throughout the three-dimensional rolled structure.

1. A composition comprising: a porous scaffold sheet of fibrous material; and living cells deposited thereupon; wherein the sheet is spirally wound in a jelly-roll like manner.

2. The composition of claim 1, comprising a plurality of the spirally wound structures.

3. The composition of claim 2, wherein the plurality of spirally wound structures are aligned substantially parallel to each other along a common axis, to form a bundle of the structures.

4. The composition of claim 3, wherein the plurality of spirally wound structures are braided, twisted or held together by a sheath.

5. The composition of claim 1, wherein the fibrous material comprises individual fibers.

6. The composition of claim 5, wherein the fibers are microfibers or nanofibers.

7. The composition of claim 1, wherein the fibrous material comprises fibers that are aligned in one direction, randomly aligned, braided, twisted, or any combination thereof.

8. The composition of claim 1, wherein the fibrous material comprises a natural fiber, a synthetic fiber, or a combination thereof.

9. The composition of claim 8, wherein the natural fiber is selected from the group consisting of collagen, fibrin, silk, thrombin, chitosan, chitin, alginic acid, hyaluronic acid, and gelatin.

10. The composition of claim 8, wherein the synthetic fiber comprises two or more polymers.

11. The composition of claim 8, wherein the synthetic fiber is selected from the group consisting of: representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate), polybutylene terephthalate (PBT), polyhydroxyhexanoate (PHH), polybutylene succinate (PBS), and poly(hydroxyl valerate).

12. The composition of claim 1, wherein the cell is selected from the group consisting of stem cells, osteoblasts, myoblasts, neuroblasts, fibroblasts, glioblasts, germ cells, hepatocytes, chondrocytes, keratinocytes, smooth muscle cells, cardiac muscle cells, connective tissue cells, epithelial cells, endothelial cells, hormone-secreting cells, neurons, tenocytes, skeletal myocytes, and skeletal myoblasts.

13. The composition of claim 12, wherein the stem cell comprises an adult stem cell, an embryonic stem cell or a reprogrammed stem cell.

14. The composition of claim 1, wherein the cell is deposited onto or into the scaffold prior to or after spirally winding of the scaffold sheet.

15. The composition of claim 1, further comprising a bioactive agent.

16. The composition of claim 15, wherein the bioactive agent comprises small molecules, proteins, compounds, drugs, synthetic agents, natural agents, polypeptides, or nucleic acids.

17. (canceled)

18. (canceled)

19. (canceled)

20. (canceled)

21. (canceled)

22. (canceled)

23. (canceled)

24. (canceled)

25. (canceled)

26. (canceled)

27. A method for replacing or enhancing a tissue, the method comprising:
(a) forming a tissue construct by contacting a scaffold sheet of fibrous material with a cell; and rolling the scaffold sheet in a jelly-roll like manner to form a spirally wound tissue construct,
(b) implanting the spirally wound tissue construct into a subject in need of tissue replacement or regeneration,
wherein the spirally wound tissue construct replaces a tissue.

28. The method of claim 27, further comprising aligning a plurality of the spirally wound tissue constructs substantially parallel to each other along a common axis, to form a bundle of the constructs prior to the implanting step.

29. The method of claim 27, wherein the plurality of spirally wound structures are braided, twisted or held together by a sheath.

30. The method of claim 27, wherein the fibrous material comprises a natural fiber, a synthetic fiber, or a combination thereof.

31. (canceled)

32. (canceled)

33. (canceled)

34. (canceled)

35. (canceled)

36. (canceled)

37. (canceled)

38. (canceled)
